(19) United States
(12) Patent Application Publication
(10) Pub. No.: US 2024/0228409 A1
(43) Pub. Date: Jul. 11, 2024

(54) METHOD FOR PRODUCING BROMOFLUOROMETHANE

(71) Applicant: Resonac Corporation, Tokyo (JP)

(72) Inventor: Kazuma MATSUI, Tokyo (JP)

(73) Assignee: Resonac Corporation, Tokyo (JP)

(21) Appl. No.: 18/562,141

(22) PCT Filed: Mar. 28, 2022
(86) PCT No.: PCT/JP2022/015079

§ 371 Date: Nov. 17, 2023

(30) Foreign Application Priority Data
May 20, 2021 (JP) 2021-085304

(51) Int. Cl. C07C 17/20; B01J 23/10; B01J 23/745; B01J 23/75; B01J 23/755; B01J 27/125; B01J 27/128

(52) U.S. Cl. CPC C07C 17/204; B01J 23/10; B01J 23/745; B01J 23/75; B01J 23/755; B01J 27/125; B01J 27/128; C07C 17/208

(57) ABSTRACT

There is provided a method for producing bromofluoromethane capable of selectively synthesizing at least one of tribromofluoromethane and dibromodifluoromethane. The method for producing bromofluoromethane includes: a fluorination step of reacting a fluorinating agent with a raw-material compound which is at least one of carbon tetrabromide and tribromofluoromethane for fluorination in the presence of a simple substance or a salt of a metal belonging to the third period or the fourth period and belonging to any of Group III to Group XIII of the periodic table to synthesize a target compound which is at least one of tribromofluoromethane and dibromodifluoromethane. The raw-material compound and the target compound are not the same.

### TECHNICAL FIELD

The present invention relates to a method for producing bromofluoromethane.

### BACKGROUND ART

Bromofluoromethane, such as tribromofluoromethane (CBr₃F) and dibromodifluoromethane (CBr₂F₂), is a high-versatile compound usable as a raw material of fluororesin, drug substances, an etching gas for producing semiconductors, and the like.

### CITATION LIST

### Patent Literature

PTL 1: CN 106278808 A

### SUMMARY OF INVENTION

### Technical Problem

Various methods for producing bromofluoromethane have been proposed (see, for example, PTL 1), but it has not been easy to selectively synthesize tribromofluoromethane or dibromodifluoromethane.

It is an object of the present invention to provide a method for producing bromofluoromethane capable of selectively synthesizing at least one of tribromofluoromethane and dibromodifluoromethane.

### Solution to Problem

To solve the above-described problem, an aspect of the present invention is as described in [1] to [9] below.
[1] A method for producing bromofluoromethane including: a fluorination step of reacting a fluorinating agent with a raw-material compound which is at least one of carbon tetrabromide and tribromofluoromethane for fluorination in the presence of a simple substance or a salt of a metal belonging to the third period or the fourth period and belonging to any of Group III to Group XIII of the periodic table to synthesize a target compound which is at least one of tribromofluoromethane and dibromodifluoromethane, in which the raw-material compound and the target compound are not the same.
[2] The method for producing bromofluoromethane according to [1], in which the fluorinating agent is an interhalogen compound having a bromine atom or an iodine atom and having three or more fluorine atoms.
[3] The method for producing bromofluoromethane according to [2], in which the interhalogen compound is at least one selected from bromine trifluoride, bromine pentafluoride, iodine pentafluoride, and iodine heptafluoride.
[4] The method for producing bromofluoromethane according to any one of [1] to [3], in which the reaction temperature of the fluorination in the fluorination step is 0° C. or more and 100° ° C. or less.
[5] The method for producing bromofluoromethane according to any one of [1] to [4], in which the simple substance of the metal is at least one selected from aluminum, scandium, iron, cobalt, and nickel.
[6] The method for producing bromofluoromethane according to any one of [1] to [4], in which the salt of the metal is at least one selected from aluminum fluoride, scandium fluoride, iron fluoride, cobalt fluoride, and nickel fluoride.
[7] The method for producing bromofluoromethane according to any one of [1] to [6], in which the amount of the simple substance or the salt of the metal is 1% by mol or more and 50% by mol or less of the amount of the raw-material compound.
[8] The method for producing bromofluoromethane according to any one of [1] to [7], the bromine atom including a plurality of bromine atoms, in which, when the target compound is synthesized by replacing one of the plurality of bromine atoms possessed by the raw-material compound with a fluorine atom, the ratio of the total molar amount of the fluorine atoms possessed by the fluorinating agent to the molar amount of the raw-material compound is set to 0.7 or more and 1.5 or less.
[9] The method for producing bromofluoromethane according to any one of [1] to [7], in which, when the raw-material compound is carbon tetrabromide, and the target compound is synthesized by replacing two of four bromine atoms possessed by the carbon tetrabromide with fluorine atoms, the ratio of the total molar amount of the fluorine atoms possessed by the fluorinating agent to the molar amount of the raw-material compound is set to 1.4 or more and 3.0 or less.

### Advantageous Effects of Invention

According to the present invention, at least one of tribromofluoromethane and dibromodifluoromethane can be selectively synthesized.

### BRIEF DESCRIPTION OF DRAWINGS

FIG. 1 is a view illustrating the structure of one example of a reactor capable of implementing a method for producing bromofluoromethane according to one embodiment of the present invention; and

FIG. 2 is a view illustrating the structure of one example of an analyzer analyzing generated bromofluoromethane.

### DESCRIPTION OF EMBODIMENTS

Hereinafter, one embodiment of the present invention will now be described. This embodiment illustrates one example of the present invention, and the present invention is not limited to this embodiment. This embodiment can be variously modified or improved, and such modified or improved aspects may also be included in the present invention.

A method for producing bromofluoromethane according to one embodiment of the present invention includes: a fluorination step of reacting a fluorinating agent with a raw-material compound which is at least one of carbon tetrabromide (CBr₄) and tribromofluoromethane (CBr₃F) for fluorination in the presence of a simple substance or a salt of a metal belonging to the third period or the fourth period and belonging to any of Group III to Group XIII of the periodic table to synthesize a target compound which is at least one of tribromofluoromethane (CBr₃F) and dibromodifluoromethane (CBr₂F₂). The raw-material compound and the target compound are not the same.

When carbon tetrabromide is fluorinated by the method for producing bromofluoromethane according to this embodiment, at least one of tribromofluoromethane and dibromodifluoromethane is obtained. When tribromofluoromethane is fluorinated by the method for producing bromofluoromethane according to this embodiment, dibromodifluoromethane is obtained. When carbon tetrabromide and tribromofluoromethane are fluorinated, there is a possibility that bromotrifluoromethane (CBrF₃) and carbon tetrafluoride (CF₄) are generated. However, even when a raw-material compound which is at least one of carbon tetrabromide and tribromofluoromethane is fluorinated by the method for producing bromofluoromethane according to this embodiment, bromotrifluoromethane and carbon tetrafluoride are hardly generated.

Thus, the method for producing bromofluoromethane according to this embodiment enables the selective (at a high selection rate) synthesis of at least one of tribromofluoromethane and dibromodifluoromethane which is the target compound. For example, the generation amounts of bromotrifluoromethane and carbon tetrafluoride which are byproducts in the fluorination step of the method for producing bromofluoromethane according to this embodiment can be individually set to less than 1% by mass or less, for example. The generation amounts of bromotrifluoromethane and carbon tetrafluoride are the ratios to the total mass of carbon tetrabromide, tribromofluoromethane, dibromodifluoromethane, bromotrifluoromethane, and carbon tetrafluoride contained in products by a fluorination reaction.

The method for producing bromofluoromethane according to this embodiment makes it possible to perform the fluorination without requiring high temperature conditions or high pressure conditions. Therefore, the method for producing bromofluoromethane according to this embodiment has advantages of being safe, consuming less energy, and having a small impact on the environment.

Tribromofluoromethane and dibromodifluoromethane have the property of generating plasma by dissociation even with weak energy and are not controlled substances under the Montreal Protocol, and therefore are suitable as an etching gas, while bromotrifluoromethane is a controlled substance under the Montreal Protocol, and thus is not usable as an etching gas. Producing carbon tetrafluoride in the method for producing bromofluoromethane according to this embodiment is not preferable because carbon tetrafluoride can be produced at lower cost in general production methods.

The method for producing bromofluoromethane according to this embodiment is described in more detail below.

### [Metal]

In the method for producing bromofluoromethane according to this embodiment, to improve the reactivity and the selection rate of the target compound of the fluorination, the fluorination is performed in the presence of the simple substance or the salt of the metal belonging to the third period or the fourth period and belonging to any of Group III to Group XIII of the periodic table. Due to the fact that the simple substance or the salt of the metal forms a complex represented by a chemical formula of the fluorinating agent present in the reaction system and [MFA]⁺[ZEB]⁻ (M represents a metal, F represents a fluorine atom, Z represents a bromine atom or an iodine atom, A and B represent optional coefficients), a compound having a specific structure is likely to be generated when the fluorination is performed, i.e., it is considered that the selectivity of the compound to be generated is improved.

Among these metals, aluminum (Al), scandium (Sc), iron (Fe), cobalt (Co), and nickel (Ni) are preferable as the simple substance of the metal from the viewpoint of availability or safety, and halides of aluminum, scandium, iron, cobalt, and nickel are preferable as the salt of the metal.

Among the halides of the metals, metal fluorides are more preferable. More specifically, aluminum fluoride (AlF₃), scandium fluoride (ScF₃), iron fluoride (FeF₂, FeF₃), cobalt fluoride (CoF₂, CoF₃), and nickel fluoride (NiF₂) are more preferable.

The simple substances of these metals may be used alone or in combination of two or more kinds thereof. The salts of the metals may be used alone or in combination of two or more kinds thereof. Further, the simple substances of the metals and the salts of the metals may be used in combination.

The amount of the simple substance or the salt of the metal to be subjected to the fluorination is not particularly limited, and preferably set to 1% by mol or more and 50% by mol or less and more preferably set to 5% by mol or more and 50% by mol or less based on the amount of the raw-material compound from the viewpoint of ease of treatment after the reaction is completed.

The shapes of the simple substance of the metal and the salt of the metal to be subjected to the fluorination are not particularly limited, and may be, for example, a film shape, a foil shape, a pellet shape, a block shape, a spherical shape, a granular shape, and a powder shape, for example. The simple substance of the metal and the salt of the metal react with the fluorinating agent to be entirely or partially converted to fluorides of the metals in some cases, but this is not a significant problem for the fluorination of the raw-material compound.

### [Fluorinating Agent]

In the method for producing bromofluoromethane according to this embodiment, the fluorinating agent is used to replace a bromine atom possessed by the raw-material compound with a fluorine atom for fluorination. As the fluorinating agent, interhalogen compounds having a bromine atom or an iodine atom and three or more fluorine atoms are preferable. From the viewpoint of availability or ease of handling, bromine trifluoride (BrF₃), bromine pentafluoride (BrF₅), iodine pentafluoride (IF₅), and iodine heptafluoride (IF₇) are more preferable. The compounds may be used alone or in combination of two or more kinds thereof.

The use amount of the fluorinating agent is preferably set to 0.7 times or more and 1.5 times or less the stoichiometric ratio. A reaction of fluorinating carbon tetrabromide to obtain dibromodifluoromethane is represented by Equation (1) below, for example. A reaction of fluorinating carbon tetrabromide to obtain tribromofluoromethane is represented by Equation (2) below, for example. In Equation (1), carbon tetrabromide reacts with (⅔) molar equivalent of bromine trifluoride to form dibromodifluoromethane. In Equation (2), carbon tetrabromide reacts with (⅓) molar equivalent of bromine trifluoride to form tribromofluoromethane.

$${CBr}_{4} + ( 2 / 3 ) {BrF}_{3} \to {CBr}_{2} {F}_{2} + ( 4 / 3 ) {Br}_{2} \qquad ( 1 )$$

$${CBr}_{4} + ( 1 / 3 ) {BrF}_{2} \to {CBr}_{3} F + ( 2 / 3 ) {Br}_{2} \qquad ( 2 )$$

Therefore, to selectively generate dibromodifluoromethane, the amount of bromine trifluoride in the fluorinating agent is preferably set to 0.7 times or more and 1.5 times or less the stoichiometric ratio (⅔ molar equivalent) in Equation (1). More specifically, when the raw-material compound is carbon tetrabromide, and the target compound is synthesized by replacing two of the four bromine atoms possessed by the carbon tetrabromide with fluorine atoms, the ratio of the total molar amount of the fluorine atoms possessed by the fluorinating agent to the molar amount of the raw-material compound is preferably set to 1.4 or more and 3.0 or less.

When the raw-material compound is carbon tetrabromide, and the target compound is synthesized by replacing two of the four bromine atoms possessed by the carbon tetrabromide with fluorine atoms, the ratio of the total molar amount of the fluorine atoms possessed by the fluorinating agent to the molar amount of the raw-material compound is preferably set to 2.0 or more and 2.8 or lee and more preferably set to 2.2 or more and 2.6 or less.

On the other hand, to selectively generate tribromofluoromethane, the amount of bromine trifluoride of the fluorinating agent is preferably set to 0.7 times or more and 1.5 times or less the stoichiometric ratio (⅓ molar equivalent) in Equation (2). More specifically, when the target compound is synthesized by replacing one of a plurality of bromine atoms possessed by the raw-material compound with a fluorine atom, the ratio of the total molar amount of the fluorine atoms possessed by the fluorinating agent to the molar amount of the raw-material compound is preferably set to 0.7 or more and 1.5 or less.

When the target compound is synthesized by replacing one of the plurality of bromine atoms possessed by the raw-material compound with a fluorine atom, the ratio of the total molar amount of the fluorine atoms possessed by the fluorinating agent to the molar amount of the raw-material compound is more preferably set to 1.0 or more and 1.4 or less and still more preferably set to 1.1 or more and 1.3 or less.

### [Fluorination Step]

The fluorination of reacting the fluorinating agent with the raw-material compound is preferably performed by a liquid phase reaction. More specifically, it is preferable to use a solvent capable of dissolving at least one of the raw-material compound and the fluorinating agent and to perform the fluorination reaction in a reaction solution obtained by dissolving at least one of the raw-material compound and the fluorinating agent in the solvent.

In the fluorination reaction of the fluorination step, the raw-material compound may be first supplied into the reaction system, and then the fluorinating agent may be supplied into the reaction system, or the raw-material compound and the fluorinating agent may be simultaneously supplied into the reaction system.

Methods for supplying the fluorinating agent into the reaction system include, for example, a method for supplying a liquid fluorinating agent or a fluorinating agent solution obtained by dissolving the fluorinating agent in a solvent into the reaction system using a dropping funnel or a mass flow controller for liquid or a method for suppling a fluorinating agent vaporized using a vaporizer into the reaction system.

The temperature of the liquid fluorinating agent or the fluorinating agent solution when the liquid fluorinating agent or the fluorinating agent solution is supplied into the reaction system may be a temperature at which the liquid fluorinating agent or the fluorinating agent solution is not solidified (e.g., temperature equal to or higher than the melting point of the fluorinating agent in the case of the liquid fluorinating agent) and at which the mass flow controller operates. In particular, from the viewpoint of controlling the fluorination reaction, the temperature of the liquid fluorinating agent or the fluorinating agent solution when the liquid fluorinating agent or the fluorinating agent solution is supplied into the reaction system is set to a temperature of about ±5° C. of the reaction temperature.

It may be acceptable that the vaporized raw-material compound or fluorinating agent is cooled and liquefied by a cooling device, and the fluorination reaction is performed while a reflux operation of returning the liquefied raw-material compound or fluorinating agent into the reaction solution is being performed.

Solvents usable for the fluorination reaction include those capable of dissolving the raw-material compound, the target compound, and the fluorinating agent and not or hardly chemically reacting with the raw-material compound, the target compound, and the fluorinating agent. The solvents include perfluoroalkanes, perfluoroethers, perfluoropolyethers, chlorofluorohydrocarbons, chlorohydrocarbons, perfluoroalkylamines, and 1-ethoxy-1,1,2,2,3,3,4,4,4-nonafluorobutane (C₆H₅F₉O), for example.

Among these solvents, perfluoroalkanes, perfluoroethers, chlorohydrocarbons, 1-ethoxy-1,1,2,2,3,3,4,4,4-nonafluorobutane are preferable and carbon tetrachloride (CCl₄), dichloromethane (CH₂Cl₂), and 1-ethoxy-1,1,2,2,3,3,4,4,4-nonafluorobutane are more preferable from the viewpoint of availability.

The amount of the solvent used for the fluorination reaction is not particularly limited, and may be determined as appropriate according to the solubility of the raw-material compound, the target compound, and the fluorinating agent in the solvent.

The reaction temperature and the reaction pressure of the fluorination reaction are not limited to any particular conditions insofar as the liquid phase reaction is possible and excessive progress of the fluorination is inhibited. The reaction temperature is preferably −80° C. or more and 200° C. or less, more preferably 0° C. or more and 100° C. or less, and still more preferably 10° C. or more and 70° C. or less. When the reaction temperature is −80° C. or more, the reaction solution is less likely to solidify, and, when the reaction temperature is 200° C. or less, the excessive progress of the fluorination is inhibited, so that the yield of the target compound increases and the target compound can be obtained at a high selection rate. The reaction pressure is preferably equal to or larger than the atmospheric pressure and 1 MPaG or less in terms of the yield and the selection rate of the target compound and ease of industrial implementation. In this specification, the pressure is indicated as the gauge pressure unless otherwise noted.

The fluorination reaction may also be performed under an inert gas atmosphere. The inert gas includes, for example, nitrogen gas (N₂), helium (He), and argon (Ar).

A reaction vessel in which the fluorination reaction is implemented may be formed of any material insofar as the material has corrosion resistance to the fluorinating agent. Suitable materials of the reaction vessel include, for example, nickel-based alloys, such as Inconel (registered trademark), Hastelloy (registered trademark), and Monel (registered trademark), nickel, aluminum, alumina, stainless steel, and platinum (Pt). The inner surfaces of the reaction vessels formed of these materials may be subjected to fluororesin lining. When the inner surfaces of the reaction vessels are subjected to the fluororesin lining, the reaction vessels may be formed of materials not having corrosion resistant to the fluorinating agent.

When the fluorination step is completed, the target compound may be taken out of the reaction solution after post-treatment is applied to the reaction solution as required. Post-treatment methods include cleaning, distillation, filtration, and the like of the reaction solution. These post-treatments may be performed alone or in combination of two or more methods thereof as appropriate.

### EXAMPLES

The present invention is more specifically described with reference to Examples and Comparative Examples.

### Example 1

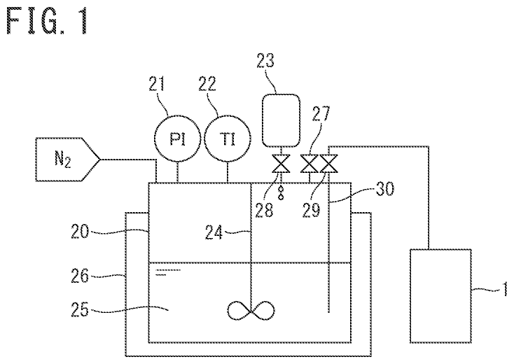

A raw-material compound was fluorinated using a reactor illustrated in FIG. 1. First, the configuration of the reactor in FIG. 1 is described. The reactor in FIG. 1 includes: a reaction vessel 20 formed of Monel (registered trademark) having a volume of 50 mL which can store a reaction solution 25 and in which the fluorination reaction of the raw-material compound is performed; a pressure gauge 21 measuring the pressure inside the reaction vessel 20; a thermometer 22 measuring the temperature of the reaction solution 25; a stirrer 24 stirring the reaction solution 25; a thermostat bath 26 controlling the temperature of the reaction solution 25; a dropping device 23 dropping a liquid fluorinating agent or a fluorinating agent solution into the reaction vessel 20; a drop rate adjustment valve 28 adjusting the drop rate of the liquid fluorinating agent or the fluorinating agent solution supplied from the dropping device 23 into the reaction vessel 20; a gas phase part extraction port 27 for extracting the gas phase part inside the reaction vessel 20; a liquid phase part extraction port 29 and a liquid phase part extraction pipe 30 for extracting the liquid phase part (reaction solution 25) inside the reaction vessel 20; and a vaporizer 1.

Next, the fluorination reaction performed using the reactor in FIG. 1 is described. A fluorinating agent solution obtained by dissolving 3.3 g (24 mmol) of bromine trifluoride in 10 mL of carbon tetrachloride was stored in the dropping device 23. In addition, 0.2 g of 99% pure nickel powder (manufactured by Hayashi Pure Chemical Ind., Ltd.), 9.9 g (30 mmol) of carbon tetrabromide, and 15 mL of carbon tetrachloride were charged into the reaction vessel 20. Then, the carbon tetrabromide was dissolved into the carbon tetrachloride by stirring with the stirrer 24 to give the reaction solution 25.

The fluorination reaction of the carbon tetrabromide was performed by dropping the fluorinating agent solution into the reaction solution 25 with the dropping device 23 while the temperature of the reaction solution 25 was being controlled to 20° C. with the thermostat bath 26. The drop rate of the fluorinating agent solution was regulated by adjusting the opening degree of the drop rate adjustment valve 28, and 10 mL of the fluorinating agent solution was dropped in one hour. After the dropping of the fluorinating agent solution was completed, the fluorination reaction was performed for another hour.

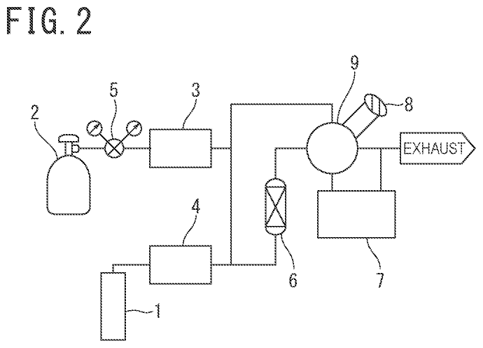

When the fluorination reaction was completed, the reaction solution 25 was allowed to stand still, and then a part of the reaction solution 25 was extracted through the liquid phase part extraction port 29 and the liquid phase part extraction pipe 30 into the vaporizer 1 in which the internal pressure was set to −0.1 MPaG in terms of gauge pressure. Then, the vaporizer 1 storing the reaction solution 25 was removed from the reactor and connected to an analyzer illustrated in FIG. 2.

Herein, the configuration of the analyzer illustrated in FIG. 2 is described. The analyzer in FIG. 2 includes: a carrier gas cylinder 2 storing carrier gas; a carrier gas pressure controller 5 controlling the pressure of the carrier gas supplied from the carrier gas cylinder 2; a carrier gas flow rate controller 3 controlling the flow rate of the carrier gas supplied from the carrier gas cylinder 2; a sample flow rate controller 4 controlling the flow rate of a sample supplied from the inside of the vaporizer 1; a fluorinating agent removal tank 6 removing the fluorinating agent from the sample supplied from the vaporizer 1; a gas gauge tube 8 measuring the gas amount; a flow path switching valve 9 switching a gas flow path; and a gas chromatograph 7 analyzing the sample supplied from the vaporizer 1 by gas chromatography.

Next, the gas chromatography performed using the analyzer in FIG. 2 is described. First, the carrier gas was supplied from the carrier gas cylinder 2 through the carrier gas pressure controller 5 and the carrier gas flow rate controller 3 to attain a state where the carrier gas circulates in the analyzer, and then the vaporizer 1 was heated in that state to vaporize the reaction solution 25 in the vaporizer 1 (hereinafter, the gas in the reaction solution 25 is referred to as “sample gas”). As the carrier gas, nitrogen gas having purity of 99.99995% or more (manufactured by TOKYO KOATSU Co., Ltd.) was used.

Next, the sample gas was sent by the carrier gas to the fluorinating agent removal tank 6 to remove the unreacted fluorinating agent from the sample gas. The inside of the fluorinating agent removal tank 6 is filled with porous nickel (specific surface area of 7500 m²/m³) manufactured by The Nilaco Corporation. The fluorinating agent removal tank 6 has an inner diameter of 1 inch and a length of 15 cm.

Subsequently, the sample gas from which the fluorinating agent was removed was made to circulate in the gas gauge tube 8, the gas amount was weighed, and then the flow path switching valve 9 was operated, thereby introducing the sample gas, from which the fluorinating agent was removed, into the gas chromatograph 7 for analysis. This determined the compositions of the components (percentage of each of carbon tetrabromide, tribromofluoromethane, dibromodifluoromethane, bromotrifluoromethane, and carbon tetrafluoride) contained in the reaction solution 25 (liquid phase part). The results are shown in Table 1.

By connecting the gas phase part extraction port 27 of the reactor, in place of the vaporizer 1, to the sample flow rate controller 4 of the analyzer, the gas phase part inside the reaction vessel 20 was extracted and introduced into the analyzer to be analyzed in the same manner as the reaction solution 25 (liquid phase part). This determined the compositions of the components contained in the gas phase part. The results are shown in Table 1.

The determination of carbon tetrabromide, tribromofluoromethane, dibromodifluoromethane, bromotrifluoromethane, and carbon tetrafluoride in the gas chromatography was performed by the absolute calibration method.

The analysis conditions of the gas chromatography are as follows.
Used equipment: Gas chromatograph GC-2014 manufactured by Shimadzu Corporation
Column: Capillary column for gas chromatography PoraPLOT (registered trademark) Q-HT manufactured by Shimadzu Corporation
Carrier gas: Nitrogen gas (flow rate: 2 mL/min)
Sample loop volume: 5 mL
Inlet temperature: 200° ° C.
Column temperature: 120° ° C.
Detector: Thermal conductivity detector (TCD)
Detector temperature: 200° ° C.
Current value: 150 mA

TABLE 1

| | Raw- | | | | | | | | | |
|---|---|---|---|---|---|---|---|---|---|---|
| | material | Fluorinating | | Simple substance | | | Reaction | | | |
| | compound | agent | | or salt of metal | | Solvent | temperature | Liquid phase composition (% by mass) | | |
| | type | Type | Amount ¹⁾ | Type | Amount ²⁾ | type | (° C.) | CBr₄ | CBr₃F | CBr₂F₂ |
| Ex. 1 | CBr₄ | BrF₃ | 2.40 | Ni | 10 | CCl₄ | 20 | Less | 3 | 97 |
| | | | | | | | | than 1 | | |
| Ex. 2 | CBr₄ | BrF₅ | 2.50 | Ni | 10 | CCl₄ | 20 | Less | 2 | 98 |
| | | | | | | | | than 1 | | |
| Ex. 3 | CBr₄ | IF₅ | 2.50 | Ni | 10 | CCl₄ | 60 | Less | 2 | 98 |
| | | | | | | | | than 1 | | |
| Ex. 4 | CBr₄ | IF₇ | 2.45 | Ni | 10 | CCl₄ | 60 | Less | 4 | 96 |
| | | | | | | | | than 1 | | |
| Ex. 5 | CBr₄ | BrF₃ | 2.40 | Ni | 10 | CH₂Cl₂ | 20 | Less | 5 | 95 |
| | | | | | | | | than 1 | | |
| Ex. 6 | CBr₄ | BrF₃ | 2.40 | Ni | 10 | 3) | 20 | Less | 6 | 94 |
| | | | | | | | | than 1 | | |
| Ex. 7 | CBr₄ | BrF₃ | 2.40 | Ni | 5 | CCl₄ | 20 | Less | 2 | 98 |
| | | | | | | | | than 1 | | |
| Ex. 8 | CBr₄ | BrF₃ | 2.40 | Ni | 20 | CCl₄ | 20 | Less | 11 | 89 |
| | | | | | | | | than 1 | | |
| Ex. 9 | CBr₄ | BrF₃ | 2.40 | Co | 10 | CCl₄ | 20 | Less | 5 | 95 |
| | | | | | | | | than 1 | | |
| Ex. 10 | CBr₄ | BrF₃ | 2.40 | Fe | 10 | CCl₄ | 20 | Less | 2 | 98 |
| | | | | | | | | than 1 | | |
| Ex. 11 | CBr₄ | BrF₃ | 2.40 | AlF₃ | 10 | CCl₄ | 20 | Less | 14 | 86 |
| | | | | | | | | than 1 | | |
| Ex. 12 | CBr₄ | BrF₃ | 2.40 | Sc | 10 | CCl₄ | 20 | Less | 13 | 87 |
| | | | | | | | | than 1 | | |
| Ex. 13 | CBr₄ | BrF₃ | 1.20 | Ni | 10 | CCl₄ | 20 | Less | 92 | 8 |
| | | | | | | | | than 1 | | |
| Ex. 14 | CBr₄ | BrF₅ | 1.25 | Ni | 10 | CCl₄ | 20 | Less | 96 | 4 |
| | | | | | | | | than 1 | | |
| Ex. 15 | CBr₄ | IF₅ | 1.25 | Ni | 10 | CCl₄ | 20 | Less | 88 | 12 |
| | | | | | | | | than 1 | | |

| | Liquid phase composition (% by mass) | | Gas phase composition (% by mass) | | | | |
|---|---|---|---|---|---|---|---|
| | CBrF₃ | CF₄ | CBr₄ | CBr₃F | CBr₂F₂ | CBrF₃ | CF₄ |
| Ex. 1 | Less | Less | Less | 1 | 99 | Less | Less |
| | than 1 | than 1 | than 1 | | | than 1 | than 1 |
| Ex. 2 | Less | Less | Less | 2 | 98 | Less | Less |
| | than 1 | than 1 | than 1 | | | than 1 | than 1 |
| Ex. 3 | Less | Less | Less | 2 | 98 | Less | Less |
| | than 1 | than 1 | than 1 | | | than 1 | than 1 |
| Ex. 4 | Less | Less | Less | 2 | 98 | Less | Less |
| | than 1 | than 1 | than 1 | | | than 1 | than 1 |
| Ex. 5 | Less | Less | Less | 3 | 97 | Less | Less |
| | than 1 | than 1 | than 1 | | | than 1 | than 1 |
| Ex. 6 | Less | Less | Less | 3 | 97 | Less | Less |
| | than 1 | than 1 | than 1 | | | than 1 | than 1 |
| Ex. 7 | Less | Less | Less | 2 | 98 | Less | Less |
| | than 1 | than 1 | than 1 | | | than 1 | than 1 |
| Ex. 8 | Less | Less | Less | 5 | 95 | Less | Less |
| | than 1 | than 1 | than 1 | | | than 1 | than 1 |
| Ex. 9 | Less | Less | Less | 3 | 97 | Less | Less |
| | than 1 | than 1 | than 1 | | | than 1 | than 1 |
| Ex. 10 | Less | Less | Less | 1 | 99 | Less | Less |
| | than 1 | than 1 | than 1 | | | than 1 | than 1 |
| Ex. 11 | Less | Less | Less | 5 | 95 | Less | Less |
| | than 1 | than 1 | than 1 | | | than 1 | than 1 |
| Ex. 12 | Less | Less | Less | 2 | 98 | Less | Less |
| | than 1 | than 1 | than 1 | | | than 1 | than 1 |
| Ex. 13 | Less | Less | Less | 10 | 90 | Less | Less |
| | than 1 | than 1 | than 1 | | | than 1 | than 1 |
| Ex. 14 | Less | Less | Less | 15 | 85 | Less | Less |
| | than 1 | than 1 | than 1 | | | than 1 | than 1 |
| Ex. 15 | Less | Less | Less | 8 | 92 | Less | Less |
| | than 1 | than 1 | than 1 | | | than 1 | than 1 |

¹⁾ Ratio of the total molar amount of fluorine atoms possessed by the fluorinating agent to the molar amount of the raw-material compound
²⁾ Ratio of the molar amount of the simple substance or the salt of metal to the molar amount of the raw-material compound
3) 1-ethoxy-1,1,2,2,3,3,4,4,4-nonafluorobutane

TABLE 2

| | Raw- | | | | | | | | | |
|---|---|---|---|---|---|---|---|---|---|---|
| | material | Fluorinating | | Simple substance | | | Reaction | | | |
| | compound | agent | | or salt of metal | | Solvent | temperature | Liquid phase composition (% by mass) | | |
| | type | Type | Amount ¹⁾ | Type | Amount ²⁾ | type | (° C.) | CBr₄ | CBr₃F | CBr₂F₂ |
| Ex. 16 | CBr₄ | IF₇ | 1.28 | Ni | 10 | CCl₄ | 20 | Less | 92 | 8 |
| | | | | | | | | than 1 | | |
| Ex. 17 | CBr₄ | BrF₃ | 1.20 | NiF₂ | 10 | CCl₄ | 20 | Less | 83 | 17 |
| | | | | | | | | than 1 | | |
| Ex. 18 | CBr₄ | BrF₃ | 1.20 | Ni | 10 | CCl₄ | 10 | Less | 95 | 5 |
| | | | | | | | | than 1 | | |
| Ex. 19 | CBr₃F | BrF₃ | 1.20 | Ni | 10 | CCl₄ | 20 | Less | 3 | 97 |
| | | | | | | | | than 1 | | |
| Ex. 20 | CBr₃F | BrF₅ | 1.25 | Ni | 10 | CCl₄ | 20 | Less | 1 | 99 |
| | | | | | | | | than 1 | | |
| Ex. 21 | CBr₃F | IF₅ | 1.25 | Ni | 10 | CCl₄ | 60 | Less | 3 | 97 |
| | | | | | | | | than 1 | | |
| Ex. 22 | CBr₃F | IF₇ | 1.28 | Ni | 10 | CCl₄ | 60 | Less | 2 | 98 |
| | | | | | | | | than 1 | | |
| Ex. 23 | CBr₃F | BrF₃ | 1.40 | Ni | 10 | CCl₄ | 20 | Less | Less | More |
| | | | | | | | | than 1 | than 1 | than 99 |
| Comp. | CBr₄ | BrF₃ | 2.40 | None | — | CCl₄ | 20 | Less | Less | 90 |
| Ex. 1 | | | | | | | | than 1 | than 1 | |
| Comp. | CBr₄ | BrF₅ | 2.50 | None | — | CCl₄ | 20 | Less | Less | 88 |
| Ex. 2 | | | | | | | | than 1 | than 1 | |
| Comp. | CBr₄ | IF₅ | 2.50 | None | — | CCl₄ | 60 | Less | 10 | 82 |
| Ex. 3 | | | | | | | | than 1 | | |
| Comp. | CBr₄ | IF₇ | 2.45 | None | — | CCl₄ | 60 | Less | 12 | 76 |
| Ex. 4 | | | | | | | | than 1 | | |
| Comp. | CBr₄ | BrF₃ | 1.20 | None | — | CCl₄ | 20 | Less | 89 | 9 |
| Ex. 5 | | | | | | | | than 1 | | |
| Comp. | CBr₃F | BrF₃ | 1.20 | None | — | CCl₄ | 20 | Less | Less | 91 |
| Ex. 6 | | | | | | | | than 1 | than 1 | |

| | Liquid phase composition (% by mass) | | Gas phase composition (% by mass) | | | | |
|---|---|---|---|---|---|---|---|
| | CBrF₃ | CF₄ | CBr₄ | CBr₃F | CBr₂F₂ | CBrF₃ | CF₄ |
| Ex. 16 | Less | Less | Less | 10 | 90 | Less | Less |
| | than 1 | than 1 | than 1 | | | than 1 | than 1 |
| Ex. 17 | Less | Less | Less | 5 | 95 | Less | Less |
| | than 1 | than 1 | than 1 | | | than 1 | than 1 |
| Ex. 18 | Less | Less | Less | 10 | 90 | Less | Less |
| | than 1 | than 1 | than 1 | | | than 1 | than 1 |
| Ex. 19 | Less | Less | Less | 1 | 99 | Less | Less |
| | than 1 | than 1 | than 1 | | | than 1 | than 1 |
| Ex. 20 | Less | Less | Less | Less | More | Less | Less |
| | than 1 | than 1 | than 1 | than 1 | than 99 | than 1 | than 1 |
| Ex. 21 | Less | Less | Less | Less | More | Less | Less |
| | than 1 | than 1 | than 1 | than 1 | than 99 | than 1 | than 1 |
| Ex. 22 | Less | Less | Less | Less | More | Less | Less |
| | than 1 | than 1 | than 1 | than 1 | than 99 | than 1 | than 1 |
| Ex. 23 | Less | Less | Less | Less | 95 | 3 | 2 |
| | than 1 | than 1 | than 1 | than 1 | | | |
| Comp. | 10 | Less | Less | Less | 81 | 14 | 5 |
| Ex. 1 | | than 1 | than 1 | than 1 | | | |
| Comp. | 12 | Less | Less | Less | 80 | 12 | 8 |
| Ex. 2 | | than 1 | than 1 | than 1 | | | |
| Comp. | 8 | Less | Less | Less | 77 | 19 | 4 |
| Ex. 3 | | than 1 | than 1 | than 1 | | | |
| Comp. | 12 | Less | Less | Less | 70 | 21 | 9 |
| Ex. 4 | | than 1 | than 1 | than 1 | | | |
| Comp. | 2 | Less | Less | 8 | 80 | 9 | 3 |
| Ex. 5 | | than 1 | than 1 | | | | |
| Comp. | 9 | Less | Less | Less | 76 | 21 | 3 |
| Ex. 6 | | than 1 | than 1 | than 1 | | | |

¹⁾ Ratio of the total molar amount of fluorine atoms possessed by the fluorinating agent to the molar amount of the raw-material compound
²⁾ Ratio of the molar amount of the simple substance or the salt of metal to the molar amount of the raw-material compound

### Examples 2 to 18

The fluorination reaction and the analysis were performed in the same manner as in Example 1, except that the type and the amount of the fluorinating agent, the type and the amount of the simple substance or the salt of the metal, the type of the solvent, and the reaction temperature in the fluorination reaction each were as shown in Tables 1, 2. The results are shown in Tables 1, 2.

### Examples 19 to 23

The fluorination reaction and the analysis were performed in the same manner as in Example 1, except that the raw-material compound was changed from carbon tetrabromide to tribromofluoromethane, and the type and the amount of the fluorinating agent and the reaction temperature in the fluorination reaction each were as shown in Table 2. The results are shown in Table 2.

### Comparative Examples 1 to 5

The fluorination reaction and the analysis were performed in the same manner as in Example 1, except that the simple substance or the salt of the metal was not used in the fluorination reaction, and the type and the amount of the fluorinating agent and the reaction temperature in the fluorination reaction each were as shown in Table 2. The results are shown in Table 2.

### Comparative Example 6

The fluorination reaction and the analysis were performed in the same manner as in Comparative Example 5, except that the raw-material compound was changed from carbon tetrabromide to tribromofluoromethane. The results are shown in Table 2.

As is understood from Table 1, Examples 1 to 12 and Comparative Examples 1 to 4 are examples in which the raw-material compound is carbon tetrabromide and the fluorinating was used in amounts of 1.20 to 1.25 times the stoichiometric ratio in the reaction of generating dibromodifluoromethane (reaction in Equation (1) above).

Further, as is understood from Tables 1, 2, Examples 13 to 18 and Comparative Example 5 are examples in which the raw-material compound is carbon tetrabromide and the fluorinating agent was used in amounts of 1.20 to 1.28 times the stoichiometric ratio in the reaction of generating tribromofluoromethane (reaction in Equation (2) above).

Further, as is understood from Table 2, Examples 19 to 23 and Comparative Example 6 are examples in which the raw-material compound is tribromofluoromethane and the fluorinating agent was used in amounts of 1.20 to 1.28 times the stoichiometric ratio in the reaction of generating dibromodifluoromethane.

Examples 1 to 4 and Examples 19 to 22 are examples in which the raw-material compound was fluorinated under conditions of using bromine trifluoride, bromine pentafluoride, iodine pentafluoride, or iodine heptafluoride as the fluorinating agent, using carbon tetrachloride as the solvent, and adding 10% by mol nickel to the raw-material compound.

When the raw-material compound was fluorinated under such conditions, dibromodifluoromethane which is the target compound was highly selectively obtained from carbon tetrabromide or tribromofluoromethane which is the raw-material compound by using the fluorinating agent in amounts of 1.20 to 1.28 times the stoichiometric ratio in the reaction of generating dibromodifluoromethane. The generation amounts of bromotrifluoromethane and carbon tetrafluoride which are not the target compounds each were less than 1% by mass.

Examples 5 and 6 are examples in which dichloromethane or 1-ethoxy-1, 1, 2, 2, 3, 3, 4, 4, 4-nonafluorobutane was used, in place of carbon tetrachloride, as the solvent. Even when the raw-material compound was fluorinated under such a condition, dibromodifluoromethane which is the target compound was selectively obtained as with Examples 1 to 4.

Examples 7 and 8 are examples in which the amount of nickel to be added was set to 5% by mol or 20% by mol. Even when the raw-material compound was fluorinated under such a condition, tribromofluoromethane and dibromodifluoromethane which are the target compounds were obtained without problems. In particular, when the amount of nickel to be added was set to 5% by mol, the ratio of dibromodifluoromethane in the compositions of the components contained in the reaction solution increased, and, when the amount of nickel to be added was set to 20% by mol, the ratio of tribromofluoromethane in the compositions of the components contained in the reaction solution increased. These facts show that the composition of the target compound to be generated can be controlled by the amount of nickel to be added.

Examples 9 to 12 are examples in which the simple substance or the salt of the metal to be added was set to aluminum fluoride, cobalt, iron, or scandium. Even when the raw-material compound was fluorinated under such a condition, dibromodifluoromethane which is the target compound was highly selectively obtained.

Examples 13 to 18 are examples in which the fluorinating agent was used in amounts of 1.20 to 1.28 times the stoichiometric ratio in the reaction of generating tribromofluoromethane. Even when the raw-material compound was fluorinated under such a condition, tribromofluoromethane which is the target compound was highly selectively obtained. In particular, the results of Example 17 show that, even when the fluoride of the metal was added, the fluorination reaction proceeds without problems and tribromofluoromethane is highly selectively obtained.

Comparative Examples 1 to 6 are examples in which the fluorination reaction was performed without adding the simple substance or the salt of the metal. When the raw-material compound was fluorinated under such a condition, at least one of bromotrifluoromethane and carbon tetrafluoride which are not the target compounds is generated as byproducts, and the composition ratio of tribromofluoromethane and dibromodifluoromethane which are the target compounds decreased.

### REFERENCE SIGNS LIST

1 vaporizer
2 carrier gas cylinder
3 carrier gas flow rate controller
4 sample flow rate controller
5 carrier gas pressure controller
6 fluorinating agent removal tank
7 gas chromatograph
8 gas gauge tube
9 flow path switching valve
20 reaction vessel
21 pressure gauge
22 thermometer
23 dropping device
24 stirrer
25 reaction solution
26 thermostat bath
27 gas phase part extraction port
28 drop rate adjustment valve
29 liquid phase part extraction port
30 liquid phase part extraction pipe

1. A method for producing bromofluoromethane comprising:
a fluorination step of reacting a fluorinating agent with a raw-material compound which is at least one of carbon tetrabromide and tribromofluoromethane for fluorination in presence of a simple substance or a salt of a metal belonging to a third period or a fourth period and belonging to any of Group III to Group XIII of a periodic table to synthesize a target compound which is at least one of tribromofluoromethane and dibromodifluoromethane, wherein
the raw-material compound and the target compound are not the same.

2. The method for producing bromofluoromethane according to claim 1, wherein the fluorinating agent is an interhalogen compound having a bromine atom or an iodine atom and having three or more fluorine atoms.

3. The method for producing bromofluoromethane according to claim 2, wherein the interhalogen compound is at least one selected from bromine trifluoride, bromine pentafluoride, iodine pentafluoride, and iodine heptafluoride.

4. The method for producing bromofluoromethane according to claim 1, wherein a reaction temperature of the fluorination in the fluorination step is 0° ° C. or more and 100° ° C. or less.

5. The method for producing bromofluoromethane according to claim 1, wherein the simple substance of the metal is at least one selected from aluminum, scandium, iron, cobalt, and nickel.

6. The method for producing bromofluoromethane according to claim 1, wherein the salt of the metal is at least one selected from aluminum fluoride, scandium fluoride, iron fluoride, cobalt fluoride, and nickel fluoride.

7. The method for producing bromofluoromethane according to claim 1, wherein an amount of the simple substance or the salt of the metal is 1% by mol or more and 50% by mol or less of an amount of the raw-material compound.

8. The method for producing bromofluoromethane according to claim 1, the bromine atom comprising a plurality of bromine atoms, wherein, when the target compound is synthesized by replacing one of the plurality of bromine atoms possessed by the raw-material compound with a fluorine atom, a ratio of a total molar amount of the fluorine atoms possessed by the fluorinating agent to a molar amount of the raw-material compound is set to 0.7 or more and 1.5 or less.

9. The method for producing bromofluoromethane according to claim 1, wherein, when the raw-material compound is carbon tetrabromide, and the target compound is synthesized by replacing two of four bromine atoms possessed by the carbon tetrabromide with fluorine atoms, a ratio of a total molar amount of the fluorine atoms possessed by the fluorinating agent to a molar amount of the raw-material compound is set to 1.4 or more and 3.0 or less.

10. The method for producing bromofluoromethane according to claim 2, wherein a reaction temperature of the fluorination in the fluorination step is 0° C. or more and 100° C. or less.

11. The method for producing bromofluoromethane according to claim 3, wherein a reaction temperature of the fluorination in the fluorination step is 0° C. or more and 100° C. or less.

12. The method for producing bromofluoromethane according to claim 2, wherein the simple substance of the metal is at least one selected from aluminum, scandium, iron, cobalt, and nickel.

13. The method for producing bromofluoromethane according to claim 3, wherein the simple substance of the metal is at least one selected from aluminum, scandium, iron, cobalt, and nickel.

14. The method for producing bromofluoromethane according to claim 4, wherein the simple substance of the metal is at least one selected from aluminum, scandium, iron, cobalt, and nickel.

15. The method for producing bromofluoromethane according to claim 2, wherein the salt of the metal is at least one selected from aluminum fluoride, scandium fluoride, iron fluoride, cobalt fluoride, and nickel fluoride.

16. The method for producing bromofluoromethane according to claim 3, wherein the salt of the metal is at least one selected from aluminum fluoride, scandium fluoride, iron fluoride, cobalt fluoride, and nickel fluoride.

17. The method for producing bromofluoromethane according to claim 4, wherein the salt of the metal is at least one selected from aluminum fluoride, scandium fluoride, iron fluoride, cobalt fluoride, and nickel fluoride.

18. The method for producing bromofluoromethane according to claim 2, wherein an amount of the simple substance or the salt of the metal is 1% by mol or more and 50% by mol or less of an amount of the raw-material compound.

19. The method for producing bromofluoromethane according to claim 3, wherein an amount of the simple substance or the salt of the metal is 1% by mol or more and 50% by mol or less of an amount of the raw-material compound.

20. The method for producing bromofluoromethane according to claim 4, wherein an amount of the simple substance or the salt of the metal is 1% by mol or more and 50% by mol or less of an amount of the raw-material compound.
